(19) United States
(12) Patent Application Publication
(10) Pub. No.: US 2007/0081962 A1
(43) Pub. Date: Apr. 12, 2007

(54) Novel delivery of immune response modifiers for removal of chronic tattoos

(76) Inventor: Amit Munshi, Thousand Oaks, CA (US)

Correspondence Address: WILSON SONSINI GOODRICH & ROSATI, 650 PAGE MILL ROAD, PALO ALTO, CA, 94304-1050, US

(21) Appl. No.: 11/245,536

(22) Filed: Oct. 6, 2005

(51) Int. Cl. A61K 38/48; A61K 8/64; A61K 31/785; A61K 38/19; A61K 38/20

(52) U.S. Cl. 424/70.14; 424/94.64; 424/78.27; 424/85.1; 424/85.2

(57) ABSTRACT

The present invention contemplates methods and compositions for removing a tattoo. In one aspect, the present invention contemplates the use of a macrophage cell disrupter, optionally with an IRM for removing a tattoo. Both the IRM and the macrophage cell disrupter may be admixed with an adhesive. In one aspect the present invention contemplates a composition comprising an adhesive and an IRM. Such composition can be found in a kit for removal of a tattoo. In any of the embodiments herein, low dose IRMs are preferred.

### INCORPORATION BY REFERENCE

All publications and patent applications mentioned in this specification are herein incorporated by reference to the same extent as if each individual publication or patent application was specifically and individually indicated to be incorporated by reference.

### SUMMARY OF THE INVENTION

The present invention relates to a method for removing a tattoo comprising administering to at least a portion of a tattooed region a composition comprising an adhesive and an immune response modulator (IRM), and optionally administering laser treatment to at least a portion of said tattooed region. Such composition can harden when exposed to air or upon contacting skin. For example, the adhesive can harden upon air or skin contact. The adhesive can comprises cyanoacrylate, or fibrin. In some embodiments, the IRM is selected from the group consisting of: imiquimod, IL-1, IL-6, and TNA-alpha.

In one aspect the present invention relates to a kit comprising: a container comprising an IRM and an adhesive; a container comprising a macrophage cell disrupter and an adhesive; and instructions for use thereof for removing a tattoo.

In one aspect, the present invention relates to a method for removing tattoo without the use of laser treatment comprising administering to at least a portion of the tattooed region a macrophage cell disrupter. The macrophage cell disrupter can be selected from the group consisting of a gas, a bacteria or bacterial product, a chemical mean, or a biological mean. The macrophage cell disrupter is selected from the group consisting of nitrous oxide, helicobacter pylori, listeria, Bacterial Redox Protein Azurin, shiga like toxins, including staphylococcal enterotoxin type B, exotoxin A and cholera toxin, and bacillas anthracis, morphine, cholesterol, P38 MAP Kinase Inhibition and oxidative low density lipoproteins. The macrophage cell disrupter can be administered with an adhesive (e.g., fibrin or cyanoacrylate).

In one aspect, the present invention relates to administering to at least a portion of the tattooed region an IRM (e.g., selected from the group consisting of IL-1, IL-6, TNF-alpha, and imiquimod). In any of the embodiments herein, the IRM is administered in a low dose. Such low dose can be, e.g., less than 10, 9, 8, 7, 6, 5, 4, 3, 2, 1, 0.9, 0.8, 0.7, 0.6, 0.5, 0.4, 0.3, 0.2, or 0.1 gram per 1-cm² of skin.

### DETAILED DESCRIPTION OF THE INVENTION

It has been found that certain immune response modulators (IRMs) can be useful in methods for removing tattoos. IRMs are compounds that possess potent immunomodulating activity such as, for example, antiviral and/or antitumor activity. Preferably, IRM's are immune response enhancers.

However, IRM's can cause an undesired inflammatory response and may be hard to apply to tattoos which are irregular in shape

Accordingly, the present invention provides a method of tattoo removal that includes administering to an irregularly shaped tattooed region an effective amount of an IRM compound. In other aspects, the present invention contemplates reduced dosages for IRM's when such IRM's are administered to a tattooed region. In other aspects the present invention contemplates methods for removing tattoos topically without electromagnetic or laser therapy to facilitate the fading and removal of chronic tattoos.

In one aspect, the present invention provides a method of removal of a tattoo that includes administering to a tattooed region an effective amount of a composition comprising: (i) one or more IRMs and (ii) one or more adhesives.

In some embodiments, an IRM is an agonist of at least one Toll-like receptor (TLR) such as, for example, TLR4, TLR7, TLR8, or TLR9.

Certain IRMs modulate the production and secretion of cytokines. For example, certain IRM compounds induce the production and secretion of cytokines such as, e.g., Type I interferons, TNF-.alpha., IL-1, IL-6, IL-8, IL-10, IL-12, MIP-1, and/or MCP-1. As another example, certain IRM compounds can inhibit production and secretion of certain TH₂ cytokines, such as IL-4 and IL-5. Additionally, some IRM compounds are said to suppress IL-1 and TNF (U.S. Pat. No. 6,518,265).

Certain IRMs are small organic molecules (e.g., molecular weight under about 1000 Daltons, preferably under about 500 Daltons, as opposed to large biological molecules such as proteins, peptides, and the like) such as those disclosed in, for example, U.S. Pat. Nos. 4,689,338; 4,929,624; 4,988,815; 5,037,986; 5,175,296; 5,238,944; 5,266,575; 5,268,376; 5,346,905; 5,352,784; 5,367,076; 5,389,640; 5,395,937; 5,446,153; 5,482,936; 5,693,811; 5,741,908; 5,756,747; 5,939,090; 6,039,969; 6,083,505; 6,110,929; 6,194,425; 6,245,776; 6,331,539; 6,376,669; 6,451,810; 6,525,064; 6,541,485; 6,545,016; 6,545,017; 6,558,951; 6,573,273; 6,656,938; 6,660,735; 6,660,747; 6,664,260; 6,664,264; 6,664,265; 6,667,312; 6,670,372; 6,677,347; 6,677,348; 6,677,349; 6,683,088; European Patent 0 394 026; U.S. Pat. Publication Nos. 2002/0016332; 2002/0055517; 2002/0110840; 2003/0133913; 2003/0199538; and 2004/0014779; and International Patent Publication Nos. WO 01/74343; WO 02/46749 WO 02/102377; WO 63/020889; WO 03/043572; WO 03/045391; and WO 03/103584.

Additional examples of small molecule IRMs include certain purine derivatives (such as those described in U.S. Pat. Nos. 6,376,501, and 6,028,076), certain imidazoquinoline amide derivatives (such as those described in U.S. Pat. No. 6,069,149), certain imidazopyridine derivatives (such as those described in U.S. Pat. No. 6,518,265), certain benzimidazole derivatives (such as those described in U.S. Pat. No. 6,387,938), certain derivatives of a 4-aminopyrimidine fused to a five membered nitrogen containing heterocyclic ring (such as adenine derivatives described in U.S. Pat. Nos. 6,376,501; 6,028,076 and 6,329,381; and in WO 02/08595), and certain 3-.beta.-D-ribofuranosylthiazolo[4,5-d]pyrimidine derivatives (such as those described in U.S. Publication No. 2003/0199461).

Other IRMs include large biological molecules such as oligonucleotide sequences. Some IRM oligonucleotide sequences contain cytosine-guanine dinucleotides (CpG) and are described, for example, in U.S. Pat. Nos. 6,194,388; 6,207,646; 6,239,116; 6,339,068; and 6,406,705. Some CpG-containing oligonucleotides can include synthetic immunomodulatory structural motifs such as those described, for example, in U.S. Pat. Nos. 6,426,334 and 6,476,000. Other IRM nucleotide sequences lack CpG sequences and are described, for example, in International Patent Publication No. WO 00/75304.

Other IRMs include biological molecules such as aminoalkyl glucosaminide phosphates (AGPs) and are described, for example, in U.S. Pat. Nos. 6,113,918; 6,303,347; 6,525,028; and 6,649,172.

One IRM compound has been shown to effective for removing freshly applied tattoos (Solis et al., Dermatol Surg. 28:83-87 (2002)). Solis et al. tattooed a group of guinea pigs with a commonly used set of tattoo inks. Topical treatment of the tattooed area with 5% irmiquimod (1-(2-methylpropyl)-1H-imidazo[4,5-c]quinolin-4-amine) cream, marketed as ALDARA (3M Pharmaceuticals, St. Paul, Minn.), was initiated within six hours of the tattoo application and continued for seven days.

In one aspect the present invention contemplates adhesives such as those that include cyanoacrylates, fibrin based adhesives, albumin gluteraldehyde type adhesives, as well as light activated adhesives.

Examples of adhesives that contain cyanoacrylate include, but are not limited to, DERMABOND (Johnson & Johnson, Inc., New Brunswick, N.J.), INDERMIL (U.S. Surgical Company, Norwalk, Conn.), GLUSTITCH (Blacklock Medical Products Inc., Canada), TISSUMEND (Veterinary Products Laboratories, Phoenix, Ariz.), VETBOND (3M Company, St. Paul, Minn.), HISTOACRYL BLUE (Davis & Geck, St. Louis, Mo.) and ORABASE SOOTHE-N-SEAL LIQUID PROTECTANT (Colgate-Palmolive Company, New York, N.Y.).

In certain embodiments, an IRM compound is mixed with an adhesive such that both are co-administered via, e.g., a topical application such as a cream, a gel, a foam, a spray, an ointment, a lotion, a solution, a suspension, an emulsion, a microemulsion, a dispersion, a paste, a powder, or an oil.

Preferably, the adhesive is fluid or liquid upon initial contact with the skin such that it (and the IRM) can be spread over various shapes of tattoos. The adhesive and the IRM can then harden to act as a patch.

When using a cyanoacrylate adhesive, both long chain (e.g., polymer of more than 10, 20, 30, 40, 50, 60, 70, 80, and 90 monomer units) and short chain cyanoacrylate (polymer of less than 10, 9, 8, 7, 6, 5, 4, 3, 2, and 1 monomer units) adhesives may be used.

In some embodiments, the present invention contemplates the compositions described above and kits comprising such compositions with instructions for use in removing a tattoo.

In any of the embodiments, a treatment regimen herein may further comprise the use of a cell disrupter, or more preferably a macrophage cell disrupter.

Examples of cell disrupters contemplated herein include topical application of mild acids, salabrasion, cryosurgery, dermabrasion, and thermal cautery methods such as, for example electrocoagulation and infrared coagulation. See, for example, Adrain et al., Clinics in Plastic Surgery, 27, 181 (2000) and Goldstein et al., J. Dermatol. Surg. Oncol. 5:901 (1979). A preferred cell disruptor for removing tattoos is a high-energy, pulsating beam of electromagnetic radiation. See, for example, Rosenberg and Gregory Clinics in Plastic Surgery, 1996; 23:2948; Anderson and Parrish, Science, 1983; 220:524-527; Wheeland, Lasers Surg. Med., 1995; 16:2-23; Zelickson et al., Lasers Surg. Med., 1994; 15:364-372; Aghassi et al., Annals of Plastic Surgery, 1999; 43:560-569; Adrain and Griffin, Aesthetic Laser Surgery, 2000; 27:181-192; and Taylor et al., The Journal of Investigative Dermatology, 1991; 97:131-136.

Suitable electromagnetic radiation may be substantially monochromatic or it may be polychromatic. In some cases, the wavelength of the electromagnetic radiation may range from about 200 nanometers to about 1300 nanometers, although some embodiments of the invention may be practiced using electromagnetic radiation having a wavelength outside this range. In some cases, the electromagnetic radiation is delivered to the tattoo region as a series of short pulses. In some cases, the length of pulse is less than one microsecond, in other cases less than 100 nanoseconds, and in still other cases less than one nanosecond.

The electromagnetic radiation may be generated in any conventional manner capable of generating an amount of energy sufficient to disrupt dermal cells. In some cases, the electromagnetic radiation is generated by a laser.

Lasers used for tattoo removal include, but are not limited to, argon lasers, carbon dioxide lasers, Er:YAG lasers, Q-switched ruby lasers, Q-switched alexandrite lasers, and Q-switched Nd:YAG lasers (Adrain et al., Clinics in Plastic Surgery, 27, 181 (2000)). Lasers that are commonly used in tattoo removal include the Q-switched Nd:YAG laser (532 nm and/or 1064 nm); Q-switched ruby laser (694 nm); and the Q-switched alexandrite laser (755 nm) (see, for example, Solis et al., Dermatol. Surg. 28:83087 (2002); and Rosenberg and Gregory, Clinics in Plastic Surgery 23(1):29-48 (1996)). In one particular embodiment, a Q-switched Nd:YAG laser (532 nm) may be used as a cell disruptor. In another embodiment, a Q-switched Nd:YAG laser (1064 nm) may be used as a cell disruptor. In another embodiment, a Q-switched alexandrite (755 nm) laser may be used as a cell disrupter. In other embodiments, a combination of lasers may be used. In certain alternative embodiments, the laser contacts the tattooed region under conditions sufficient to disrupt dermal cells but inadequate to disrupt all or many of the pigment particles. In certain embodiments, the laser used is sufficient to disrupt macrophages but not other cells.

Examples of macrophage cell disrupters that are contemplated herein include, but are not limited to gases (e.g., nitrous oxide), bacteria and/or bacterial byproducts (e.g., helicobacter pylori, listeria, bacterial redox protein azurin, shiga like toxin, stapholyococcal enterotoxin type B, exotoxin A, cholera toxin, and bacillas anthracis) and other chemical and biological means include, but not limited to, morphine, cholesterol, p38 MAP kinase inhibition, and oxidative low density lipoproteins. In any of the embodiments herein, macrophage cell disrupters are preferably non-electromagentic or non-laser treatments. Preferably, macrophage cell disrupters are biological or chemical treatments.

In one aspect, the present invention contemplates administering a macrophage cell disrupter to a tattooed region with an IRM modulator (IL-1, IL-6, TNF-alpha, imiquimod).

In one aspect, the present invention contemplates administering an IRM and a macrophage cell disrupter in alternating pattern to a tattooed region. Such alternation can occur hourly, twice a day, three times a day, four times a day, daily, biweekly, weekly, bimonthly, monthly, etc. For example, an IRM patch (e.g., cyanoacrylate adhesive, which forms a patch) can be administered to a tattooed region for a period of time, removed, and a second patch (e.g., cyanoacrylate adhesive, which forms a patch) which also comprises a macrophage cell disrupter (e.g., a bacteria or bacterial product) can be administered to the same tattooed region. The above steps are repeated more than once, twice, 3, 4, 5, 6, 7, 8, 9, or 10 time, or until a tattoo is partially or fully removed.

The IRMS and/or the macrophage cell disrupters can be delivered from e.g., gels, glues, patches, etc and will provide a chemical and biological process whereby a chronic tattoo can be effectively removed. Preferably, the IRMs and/or macrophage cell disrupters are administered with an adhesive. Thus, in some embodiments, the present invention contemplates a kit comprising a first container containing an IRM and an adhesive and a second container containing a macrophage cell disrupter and an adhesive. Such kits can further include a set of instructions for use thereof for removing a tattoo.

In any of the embodiments, a treatment regimen does not consist of an electromagnetic radiation treatment. In any of the embodiments, a treatment regimen does not consist of a laser treatment.

In certain embodiments, treatment with a cell disrupter takes place after the administration of an IRM compound. In certain embodiments, treatment with a cell disrupter takes place coincident with the administration of an IRM compound.

In order to avoid harmful effects of the IRM's the present invention, the present invention contemplates low dose IRM's. Therefore, in any of the embodiments herein, the dose of an IRM can be less than 5%, 4.5%, 4%, 3.5%, 3.0%, 2.5%, 2.0%, 2.5%, 1.0%, 0.5% IRM (e.g., imiquimod). In some embodiments, the dosage of the IRM is the composition is 0.1-5%, 1-4.5%, 1.5-4%, 2-3.5%, or 2.5-3%. In some embodiments, less than 10, 9, 8, 7, 6, 5, 4, 3, 2, 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01 grams of IRM is administered per 1-cm² of skin.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for removing a tattoo comprising administering to at least a portion of a tattooed region a composition comprising an adhesive and an immune response modulator (IRM), and optionally administering laser treatment to at least a portion of said tattooed region.

2. The method of claim 1 wherein said composition hardens when exposed to air or upon contacting skin.

3. The method of claim 1 wherein said adhesive comprises cyanoacrylate.

4. The method of claim 1 wherein said adhesive comprises fibrin.

5. The method of claim 1 wherein said (IRM) is selected from the group consisting of: imiquimod, IL-1, IL-6, and TNA-alpha.

6. A composition comprising an adhesive that hardens upon air or skin contact and an IRM.

7. The composition of claim 6 wherein said IRM is selected from the group consisting of: imiquimod, IL-1, IL-6, and TNA-alpha.

8. The composition of claim 6 wherein said adhesive comprises cyanoacrylate or fibrin.

9. A kit comprising:
a container comprising an IRM and an adhesive;
a container comprising a macrophage cell disrupter and an adhesive; and
instructions for use thereof for removing a tattoo.

10. A method for removing tattoo without the use of laser treatment comprising administering to at least a portion of the tattooed region a macrophage cell disrupter.

11. The method of claim 10 wherein said macrophage cell disrupter is selected from the group consisting of a gas, a bacteria or bacterial product, a chemical mean, or a biological mean.

12. The method of claim 10 wherein said macrophage cell disrupter is selected from the group consisting of nitrous oxide, helicobacter pylori, listeria, Bacterial Redox Protein Azurin, shiga like toxins, including staphylococcal enterotoxin type B, exotoxin A and cholera toxin, and bacillas anthracis, morphine, cholesterol, P38 MAP Kinase Inhibition and oxidative low density lipoproteins.

13. The method of claim 10 wherein said macrophage cell disrupter is administered with an adhesive.

14. The method of claim 13 wherein said adhesive comprises fibrin.

15. The method of claim 13 wherein said adhesive comprises cyanoacrylate.

16. The method of claim 10 wherein said method further comprises administering to at least a portion of the tattooed region an IRM.

17. The method of claim 16 wherein said IRM is selected from the group consisting of IL-1, IL-6, TNF-alpha, and imiquimod.

18. The method of claim 16 wherein said step of administering an IRM and said step of administering said macrophage cell disrupter occur in an alternating pattern.

19. The method of claim 16 wherein said IRM and said macrophage cell disrupter are delivered with an adhesive.

20. The method of claim 19 wherein said adhesive comprises fibrin.

21. The method of claim 19 wherein said adhesive comprises cyanoacrylate.
